(19) United States
(12) Patent Application Publication
(10) Pub. No.: US 2018/0214136 A1
(43) Pub. Date: Aug. 2, 2018

(54) Allergy Skin Testing Devices with Rigid Annular Pain-Reduction Structures Supported by Respective Compressible Sleeves

(71) Applicants: Richard Caizza, Union City, NJ (US); Ziv Harish, Tenafly, NJ (US); Russ Weinzimmer, Milford, NH (US)

(72) Inventors: Richard Caizza, Union City, NJ (US); Ziv Harish, Tenafly, NJ (US); Russ Weinzimmer, Milford, NH (US)

(21) Appl. No.: 15/796,104

(22) Filed: Oct. 27, 2017

Related U.S. Application Data
(63) Continuation-in-part of application No. 15/419,738, filed on Jan. 30, 2017.

(51) Int. Cl. A61B 10/00; A61B 5/00

(52) U.S. Cl. CPC A61B 10/0035; A61M 37/00; A61B 5/00; A61B 5/411

(57) ABSTRACT

Single and multiple allergy skin testing devices with rigid annular pain-reduction structures supported by respective compressible sleeves. Each rigid annular pain-reduction structure presses against skin before and while at least one corresponding sharp probe presses against the skin surrounded by the rigid annular pain-reduction structure, thereby causing a surrounding pain gate effect to reduce perceived pain due to the at least one sharp probe. Each allergy skin testing device includes: a central post having a sharp portion, a longitudinal portion, and a guide portion; a compressible structure having a leading portion, a resilient compressible portion, and a base portion; and a rigid annular probe portion having an annular skin-contacting surface. The surrounding pain gate effect is activated when the annular skin-contacting surface is pressed against the skin before and while the one or more sharp probes are pressed against the skin surrounded by the annular skin-contacting surface to administer allergen.

### CROSS-REFERENCE TO RELATED APPLICATIONS

This application is a Continuation-in-Part of application Ser. No. 15/419,738, filed Jan. 30, 2017, entitled “Allergy Skin Testing Devices with Compressible Annular Pain-Reduction Structures”, the entire contents of which are herein incorporated by reference.

### FIELD OF THE INVENTION

This invention relates generally to allergy skin testing devices, and particularly to allergy skin testing devices with structures to reduce pain.

### BACKGROUND OF THE INVENTION

Traditional allergy skin testing devices inflict pain while introducing allergens to the skin using sharp probes. According to pain-gate theory, applying dull pressure shortly before the sharp probes penetrate the skin to introduce the allergens reduces perception of the pain caused by the sharp probes. Reduced-pain allergy skin testing devices employing pain-gate theory to allergy skin testing were first taught by Harish et al. For example, Harish et al first taught using a single dull probe for each plurality of sharp probes. Heine et al then taught a version of this approach using a single dull probe for each plurality of surrounding sharp probes. However, each single dull probe of Heine et al does not provide an acceptable level of pain reduction for the corresponding plurality of surrounding sharp probes.

### SUMMARY OF THE INVENTION

According to the invention, an annular surface of a rigid annular pain-reduction structure supported by a respective compressible sleeve is pressed against the skin before one or more sharp probes is pressed against the skin surrounded by the annular surface so as to deliver an allergen to the skin for testing purposes. The pain typically caused by the one or more sharp probes is blocked by the pain gating effects of the surrounding skin contact and pressure caused by the rigid annular pain-reduction structure supported by the respective compressible sleeve. The rigid annular pain-reduction structure supported by the respective compressible sleeve provides a surround-effect that creates a plurality of pain gates substantially surrounding an area of sharp probe penetration that would be painful if not blocked by the surrounding pain gates.

The plurality of surrounding pain gates thereby induced is referred to as a PAIN FENCE™, and the annular surface of a rigid annular pain-reduction structure supported by a respective compressible sleeve to be pressed against the skin is an example of PAIN FENCE™ pain reduction technology. Thus, a single dull probe pain-reduction structure cannot induce a PAIN FENCE™.

The rigid annular pain-reduction structure supported by a respective compressible sleeve also tends to visually hide the one or more sharp probes surrounded thereby, as like being hidden behind a fence, which makes the skin testing device of the invention less intimidating, and consequently less upsetting to children.

Further, by avoiding upsetting a child to be tested by the device, pain perception is further reduced beyond the physiological “pain gate” effect caused by the surrounding rigid annular pain-reduction structure supported by a respective compressible sleeve.

In addition, the physician or nurse administering the test can suggest to the patient to expect to feel a dull pressure, such as by saying “you will feel a dull pressure”. Since the patient only sees the rigid annular pain-reduction structure about to contact the skin (which effectively hides the sharp probes inside), and has been told to expect to “feel a dull pressure”, or similar expression, he/she fully expects to feel dull pressure, and so is even less likely to experience any discomfort or distress. Thus, the allergy skin testing device of the invention provides significantly enhanced patient comfort over all known skin testing devices.

A general aspect of the invention is an allergy skin testing device, the device to be used by pressing the device against skin. The allergy skin testing device includes: a central post having a sharp portion, a longitudinal portion, and a guide portion, the sharp portion having one or more sharp probes extending longitudinally and being configured to administer an allergen to the skin, the guide portion being configured to facilitate guiding the sharp portion towards the skin, the longitudinal portion extending between the sharp portion and the guide portion; a compressible structure having a leading portion surrounding the sharp portion of the central post, a resilient compressible portion, and a base portion, the resilient compressible portion being configured to compress along the longitudinal portion of the central post while the base portion is urged towards the leading portion by the guide portion, the leading portion having a first passageway to allow passage of the sharp portion therethrough; and a rigid annular probe portion having an annular skin-contacting surface, and a second passageway extending through the rigid annular probe portion, the rigid annular probe portion extending longitudinally beyond the sharp portion of the central post, before the resilient compressible portion is compressed, the second passageway allowing passage of the sharp portion therethrough, while the resilient compressible portion is compressed, the annular skin-contacting surface being configured to activate a surrounding pain gate effect when pressed against the skin before and while the one or more sharp probes are pressed against the skin so as to administer the allergen to the skin.

In some embodiments, the resilient compressible portion of the compressible structure includes: a plurality of bendable elements configured to resiliently bend when compressed.

In some embodiments, the resilient compressible portion of the compressible annular pain-reduction structure includes: a plurality of bendable elements configured to resiliently bend outwards when compressed.

In some embodiments, an inner diameter of the first passageway is substantially similar to an inner diameter of the second passageway.

In some embodiments, an outer diameter of the annular skin-contacting surface is greater than an outer diameter of the leading portion of the compressible structure.

In some embodiments, an outer diameter of the annular skin-contacting surface is substantially similar to an outer diameter of the leading portion of the compressible structure.

In some embodiments, the annular skin-contacting surface includes: one or more bumps configured to contact the skin so as to activate a surrounding pain gate effect when pressed against the skin.

In some embodiments, the annular skin-contacting surface of the rigid annular probe portion includes: surface features that depart from being flat.

In some embodiments, the annular skin-contacting surface of the rigid annular probe portion includes a convex surface of revolution defined by rotating a generatrix curve about a central axis of the rigid annular pain-reduction structure, the generatrix curve being one of: a semi-circle, a semi-ellipse, a convex half sine curve, a convex spline curve.

In some embodiments, the annular skin-contacting surface of the rigid annular probe portion includes: a half ring torus surface as the annular skin-contacting surface, thereby providing a rounded end of the of the rigid annular probe portion having enhanced dullness to augment the surrounding pain gate effect of the rigid annular probe portion.

Another general aspect of the invention is a multiple test allergy skin testing device, the device to be used by pressing the device against skin. The multiple test allergy skin testing device includes: a plurality of central posts, each central post having a sharp portion, a longitudinal portion, and a guide portion, the sharp portion having one or more sharp probes extending longitudinally and being configured to administer an allergen to the skin, the guide portion being configured to facilitate guiding the sharp portion towards the skin, the longitudinal portion extending between the sharp portion and the guide portion; a plurality of compressible structures, each compressible structure having a leading portion surrounding the sharp portion of the central post, a resilient compressible portion, and a base portion, the resilient compressible portion being configured to compress along the longitudinal portion of the central post while the base portion is urged towards the leading portion by the guide portion, the leading portion having a first passageway to allow passage of the sharp portion therethrough; a plurality of rigid annular probe portions, each rigid annular probe portion having an annular skin-contacting surface, and a second passageway extending through the rigid annular probe portion, the rigid annular probe portion extending longitudinally beyond the sharp portion of the central post, before the resilient compressible portion is compressed, the second passageway allowing passage of the sharp portion therethrough, while the resilient compressible portion is compressed, the annular skin-contacting surface being configured to activate a surrounding pain gate effect when pressed against the skin before and while the one or more sharp probes are pressed against the skin so as to administer the allergen to the skin; and a gripping element, connected to each guide portion of each of the plurality of central posts, the gripping element being for pressing each rigid annular probe portion against the skin, and then pressing the sharp portion of each respective central post against the skin, using one press of the gripping element.

In some embodiments, the resilient compressible portion of each compressible structure includes: a plurality of bendable elements configured to resiliently bend when compressed.

In some embodiments, the resilient compressible portion of each compressible structure includes: a plurality of bendable elements configured to resiliently bend outwards when compressed.

In some embodiments, an inner diameter of each first passageway is substantially similar to an inner diameter of each respective second passageway.

In some embodiments, an outer diameter of each annular skin-contacting surface is greater than an outer diameter of the leading portion of each respective compressible structure.

In some embodiments, an outer diameter of each annular skin-contacting surface is substantially similar to an outer diameter of each respective leading portion of the compressible structure.

In some embodiments, each annular skin-contacting surface includes one or more bumps configured to contact the skin so as to activate a surrounding pain gate effect when pressed against the skin.

In some embodiments, each annular skin-contacting surface of each rigid annular probe portion includes: surface features that depart from being flat.

In some embodiments, each annular skin-contacting surface of each rigid annular probe portion includes a convex surface of revolution defined by rotating a generatrix curve about a central axis of the rigid annular pain-reduction structure, the generatrix curve being one of: a semi-circle, a semi-ellipse, a convex half sine curve, a convex spline curve.

In some embodiments, each annular skin-contacting surface of each rigid annular probe portion includes: a half ring torus surface as the annular skin-contacting surface, thereby providing a rounded end of the of the rigid annular probe portion having enhanced dullness to augment the surrounding pain gate effect of the rigid annular probe portion.

### BRIEF DESCRIPTION OF THE DRAWINGS

The invention will be more fully understood from the following detailed description, in conjunction with the following figures, wherein:

FIG. 1 is an isometric view of an embodiment of the allergy skin testing device.

FIG. 2 is a side view of the allergy skin testing device of FIG. 1.

FIG. 3 is a cross-sectional side view of the allergy skin testing device of FIGS. 1 and 2.

FIG. 4 is an isometric view of a plurality of the allergy skin testing device of FIGS. 1, 2, and 3 joined together by a handle so as to form a multiple test allergy skin testing device.

FIG. 5 is a bottom view of the multiple test allergy skin testing device of FIG. 4.

FIG. 6 is an isometric view of the multiple test allergy skin testing device pressed against a skin surface and responding to downward pressure on the handle by entering a compressed state.

FIG. 7 is an isometric view of the multiple test skin testing device of FIG. 6 in a compressed state, showing sharps probes extending through and surrounded by each annular dull probe.

FIG. 8 is an isometric view of the allergy skin testing device of FIG. 1 in a compressed state.

FIG. 9 is an exploded view of the allergy skin testing device of FIG. 1.

FIG. 10 is an isometric view of an alternate embodiment of the skin testing device having a resilient compressible portion with only a single bendable element.

FIG. 11 is an isometric view of the alternate embodiment of the skin testing device of FIG. 10 in a compressed state, showing the sharps probes extending through and surrounded by an annular surface of the annular dull probe.

FIG. 12 is an isometric view of yet another alternate embodiment of the skin testing device having a slotted annular dull probe, the slotted annular dull probe having one or more contact bumps.

FIG. 13 is an isometric view of still another alternate embodiment of the skin testing device having a bellows as the compression member.

FIG. 14 is a translucent isometric view of the embodiment of FIG. 13.

FIG. 15 is a translucent isometric view of still another alternate embodiment of the skin testing device having a spring included in the compression member.

FIG. 16 is a side view of the embodiment of the allergy skin testing device of FIG. 2, further including a stabilizing sealing ring.

FIG. 17 is a translucent isometric view of the embodiment of the skin testing device of FIG. 10 having a resilient compressible portion with only a single bendable element, also including a pair of stabilizing sealing rings.

FIG. 18 is a cross-sectional side view of yet another alternate embodiment of the skin testing device having a spring and a cover that extends over the spring.

FIG. 19 is a side view of the embodiment of FIG. 18, showing the annular dull probe contacting the skin before downward force is applied to the guide portion.

FIG. 20 is an isometric view of an embodiment of a single allergy skin testing device having a rigid annular pain-reduction structure supported by a compressible sleeve.

FIG. 21 is a wire-frame isometric view of the allergy skin testing device of FIG. 20.

FIG. 22 is a cut-away isometric view of the allergy skin testing device of FIG. 20.

FIG. 23 is a cut-away side view of the allergy skin testing device of FIG. 20.

FIG. 24 is an isometric view of a multiple test allergy skin testing device including a plurality of allergy skin testing devices of FIG. 20.

### DETAILED DESCRIPTION

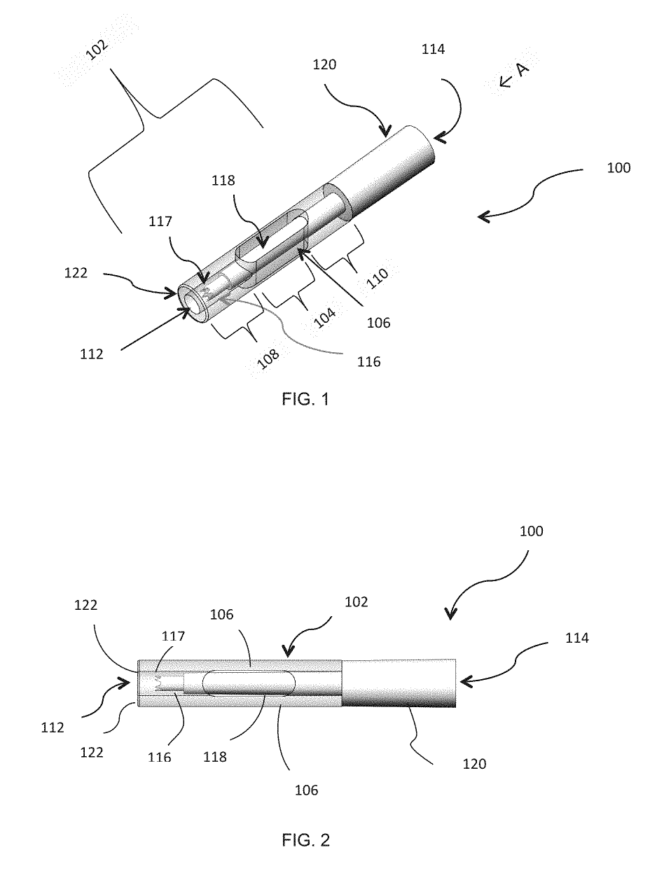

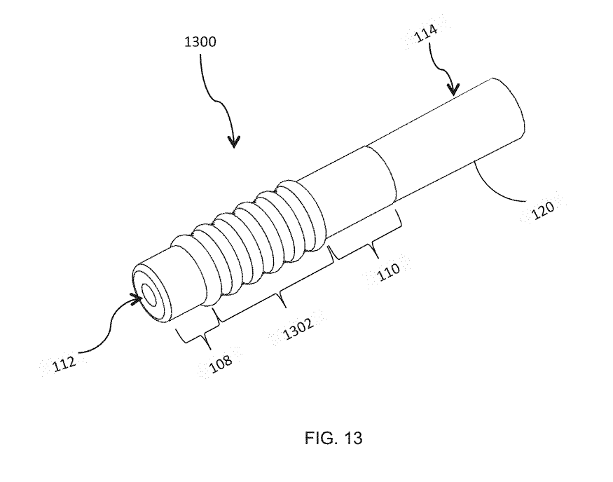

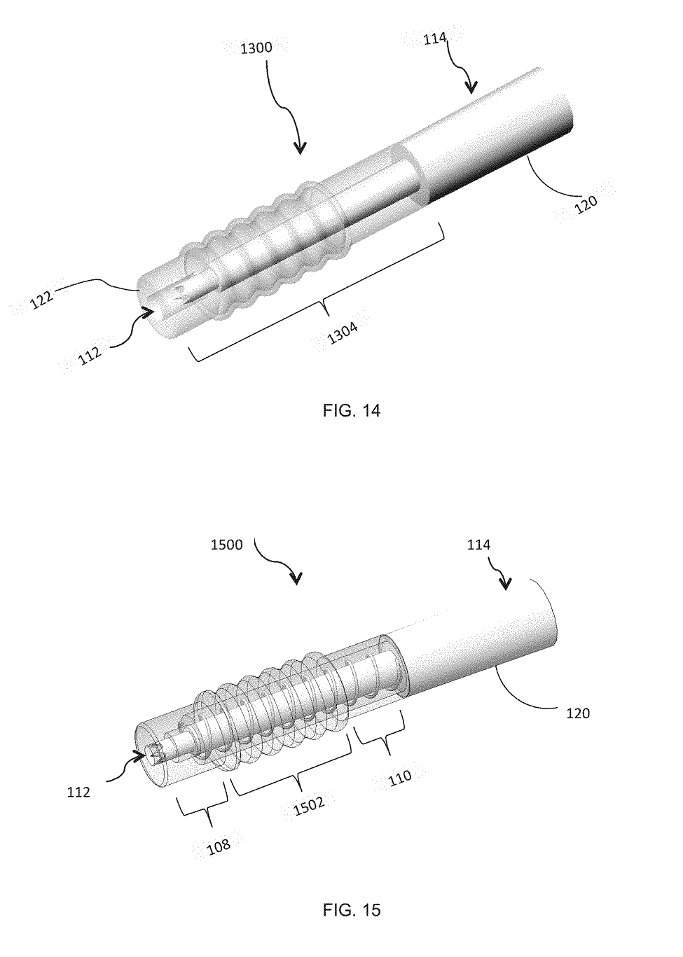

With reference to FIGS. 1 and 2, an embodiment of an allergy skin testing device 100 is shown having a compressible annular pain-reduction structure 102. In this embodiment 100, the resilient compressible portion 104 of the compressible annular pain-reduction structure 102 includes a pair of bendable elements 106 configured to resiliently bend when compressed. Note that the pair of bendable elements 106 could also be three, four, or five bendable elements, for example. The compressible annular pain-reduction structure 102 also includes an annular dull probe portion 108, and a base portion 110. The compressible annular pain-reduction structure 102 can be made from a simple extruded tube-like structure, such as shown in FIGS. 1-12, or can be a bellows-like structure, such as shown in FIGS. 13-14. The compressible annular pain-reduction structure 102 can be made of a thermoplastic, natural or synthetic rubber, or metal, for example.

Extending through a passageway 112 of the compressible annular pain-reduction structure 102 is a central post 114 having a sharp portion 116, a longitudinal portion 118, and a guide portion 120. The sharp portion 116 has one or more sharp probes 117 extending longitudinally and being configured to administer an allergen to the skin. The guide portion 120 is configured to facilitate guiding the sharp portion 116 towards the skin. The longitudinal portion 118 extends between the sharp portion 116 and the guide portion 120. The central post can be made from plastic, composite, or other light-weight material.

The annular dull probe portion 108 initially extends beyond the sharp portion 116 of the central post 114, and has a passageway 112 that allows passage of the sharp portion 116 therethrough when the guide portion 120 is pressed towards the skin. The annular dull probe portion 108 is configured to activate a surrounding pain gate effect (also called a Pain Fence™) when the annular surface 122 of the annular dull probe portion 108 is pressed against the skin.

The resilient compressible portion 104 is configured to compress along the longitudinal portion of the central post 114 while the annular surface 122 of the annular dull probe portion 108 is pressed against the skin. Thus, the resilient compressible portion 104 provides a pushing force to the annular dull probe portion 108 while the sharp probes 117 of the sharp portion 116 penetrate the skin enough to introduce the allergen that adheres to the sharp probes 117.

The base portion 110 of the compressible annular pain-reduction structure 102 is configured to be pushed by the guide portion 120 of the central post 114 when the annular dull probe portion 108 is pressed against the skin, thereby compressing the resilient compressible portion 104 until the one or more sharp probes 117 are pressed into the skin so as to administer the allergen to the skin. As the base portion 110 is pulled away from the skin by the guide portion 120 of the central post 114, the resilient compressible portion 104 expands, the sharp probes 117 move away from the skin, and then the annular dull probe portion 108 moves away from the skin.

In a preferred embodiment, reduction of pain was demonstrated when annular dull probe portion 108 extended beyond the sharp probes 117 by 1/16 inch, and the pair of bendable elements 106 resisted buckling until a force of 1-2 pounds was applied. The annular surface 122 of the annular dull probe portion 108 measured ¼ inch in diameter, with a wall thickness of 1/16 inch. The wall thickness is uniform all along the compressible annular pain-reduction structure 102, from the annular surface 122 of the annular dull probe portion 108, all the way to the base portion 110, including the bendable elements 106.

In this preferred embodiment, the bendable elements 106 are ½ inch long by 1/16 inch wide. As the bendable elements 106 buckle, or bend outward, the sharp probes 117 move through the passageway 112 to make contact with the skin, delivering the allergen to be tested thereto.

In this preferred embodiment, the material used for the bendable elements 106 is silicone with a hardness between 30-40 Shore A Durometer. The material tested was between 30-40 Shore A Durometer. “Shore A Durometer” is a hardness scale used to quantify hardness of rubber and plastics. Durometer is a dimensionless quantity.

Other materials can also be used, such as thermoplastic elastomer (TPE), polyurethane (PU), polyethylene (PE), natural rubber, or synthetic rubber, for example.

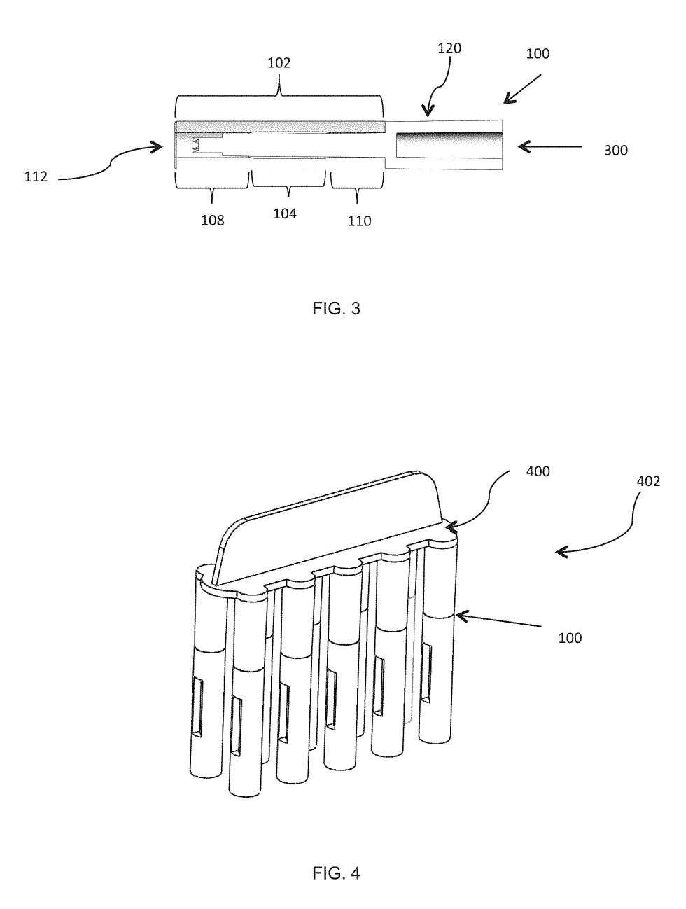

FIG. 3 shows a cross-sectional side view of the allergy skin testing device of FIGS. 1 and 2, further showing the guide portion 120 having a slot 300 which can serve as an attachment feature for attaching the guide portion 120 to a handle of a single allergy testing device, or to a handle 400 of multiple allergy testing device 402 having ten allergy testing devices 100, for example as shown in FIG. 4.

FIG. 4 shows a plurality of the allergy skin testing devices 100 of FIG. 1, joined together by a handle 400 so as to form a multiple allergy skin testing device 402.

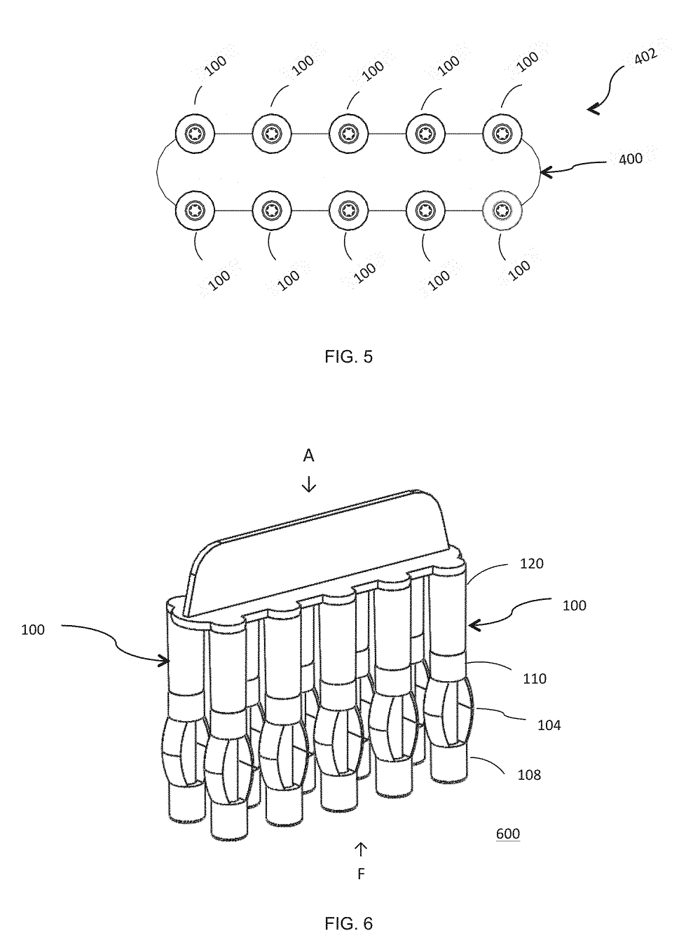

FIG. 5 shows a view from below of the plurality of the allergy skin testing devices 100 of FIG. 1, joined together by a handle 400 so as to form a multiple allergy skin testing device 402.

FIG. 6 shows the multiple allergy skin testing device 402 being pressed downward against skin 600 with force A applied to the handle 400, causing a reacting force F upward from the skin 600 as the device 402 is pressed against the skin 600 of a person being tested for various allergies. The force A is applied until the sharp probes 117 press into the skin 600 enough to transfer some allergen to the skin 600. Note that the sharp probes 117 cannot be seen by the person being tested because the sharp probes 117 are blocked from view by the annular dull probe portion 108. Also note that the resilient compressible portion 104 bows outward for each device 100 when the force A is applied to the handle 400. The handle 400 applies the force A to both the guide portion 120, and to the base portion 110. The annular dull probe portion 108 presses against the skin 600 while the sharp probes 117 press into the skin 600, the pressure of the annular dull probe portion 108 causing a surrounding pain gate effect in the nerves surrounding the points within receiving the sharp probes 117, substantially reducing the perception of pain due to the sharp probes 117.

When force A is reversed, as when the device 402 is pulled away from the skin 600, the sharp probes 117 retreat back into the passageway 112 of the annular dull probe portion 108. The annular dull probe portion 108 shields the sharp probes 117 from sight, thereby reducing anxiety of the patients who may fear sharp probes.

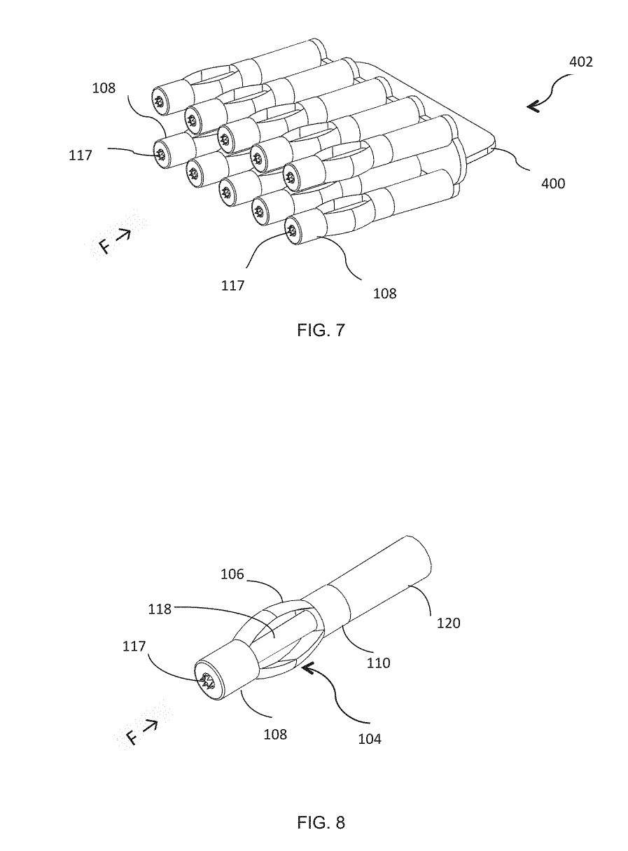

FIG. 7 shows a bottom and side view of the device 402 of FIG. 6, also in the compressed state, but now showing the sharp probes 117 emerging from the passageway 112 of each annular dull probe portion 108.

FIG. 8 shows a single tester device 100 of the multiple allergy skin testing device 402 of FIG. 6, also in a compressed state due to application of the force F. The sharp probes 117 can be seen extending beyond the annular dull probe portion 108 through the passageway 112. The bendable elements 106 of the resilient compressible portion 104 are also seen flanking the longitudinal portion 118 that extends between the sharp portion 116 supporting the sharp probes 117 and the guide portion 120.

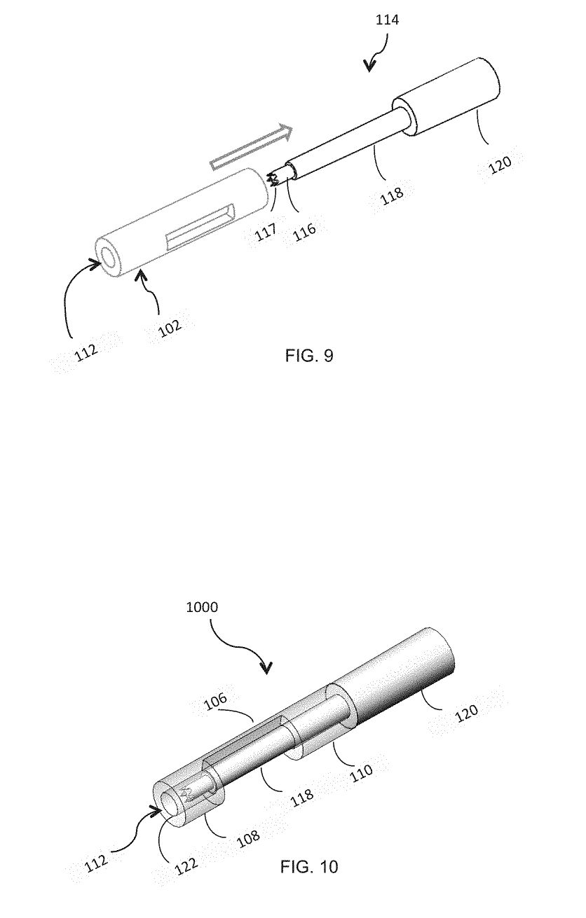

FIG. 9 is an exploded view of the allergy skin testing device of FIG. 1, showing the central post 114 having a sharp portion 116, a longitudinal portion 118, and a guide portion 120. The sharp portion 116 has one or more sharp probes 117 extending longitudinally and is configured to administer an allergen to the skin. The guide portion 120 is configured to facilitate guiding the sharp portion 116 towards the skin. The longitudinal portion 118 extends between the sharp portion 116 and the guide portion 120. The sharp portion 116 and the longitudinal portion 118 extend through most of the compressible annular pain-reduction structure 102 while the compressible annular pain-reduction structure 102 is not compressed, as shown in FIG. 2.

FIG. 10 is an isometric view of an alternate embodiment 1000 of the skin testing device having an asymmetric resilient compressible portion 104. In this embodiment 1000, the resilient compressible portion 104 has only a single bendable element 106 configured to resiliently bend when compressed.

FIG. 11 shows the alternate embodiment 1000 of FIG. 10 in a compressed state, showing the sharps probes 117 extending through and surrounded by the annular surface 122 of the annular dull probe portion 108.

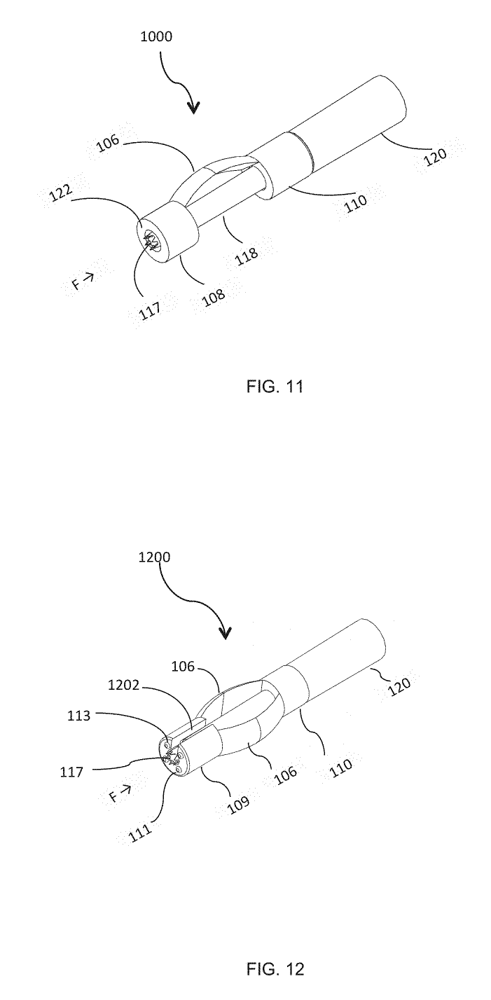

FIG. 12 is an isometric view of yet another alternate embodiment 1200 of the skin testing device having a slotted annular dull probe portion 109. A slot 1202 runs longitudinally along the slotted annular dull probe portion 109. The annular surface 111 of the slotted annular dull probe portion 109 has one or more bumps 113. Thus, the annular dull probe portion 109 need not fully surround the sharp probes to provide a surface for providing a surrounding pain gate effect. Further, the annular surface 111 of the annular dull probe portion 109 that contacts a patient's skin may include surface features, such as bumps 113 or ridges, to distract the patient from any sensation of the sharp probes 117 penetrating the skin, and to enhance the surrounding pain gate effect.

FIG. 13 is an isometric view of still another alternate embodiment 1300 of the skin testing device having an accordion resilient compressible portion 1302 between the annular dull probe portion 108, and the base portion 110. In this view, the guide portion 120 of the central post 114 is visible. Note that the sharp portion 116 is not visible within the passageway 112.

FIG. 14 is a translucent isometric view of the embodiment of FIG. 13. In this view, the entire central post 114 is visible, including the sharp portion 116 and the longitudinal portion 118. The sharp portion 116 and the longitudinal portion 118 extend through the entire compressible annular pain-reduction structure 1304, and will extend beyond the annular surface 122 and out from the passage way 112 when the resilient compressible portion 1302 is compressed.

FIG. 15 is a translucent isometric view of still another alternate embodiment 1500 of the skin testing device having a spring resilient compressible portion 1502 between the annular dull probe portion 108, and the base portion 110. In this view, the guide portion 120 of the central post 114 is visible. Note that the sharp portion 116 is visible within the passageway 112, and will emerge from the passageway 112 when the spring resilient compressible portion 1502 is compressed. The spring resilient compressible portion 1502 can include helical metal wire, helical plastic, or other resilient material. The spring resilient compressible portion 1502 can extend through the base portion 110. The spring resilient compressible portion 1502 can include an optional bellows cover portion like the bellows 1302 in FIG. 13. The annular dull probe portion 108, and the base portion 110 can be made from a thermoplastic, medical-grade plastic, a natural or synthetic rubber, silicone, or metal, for example.

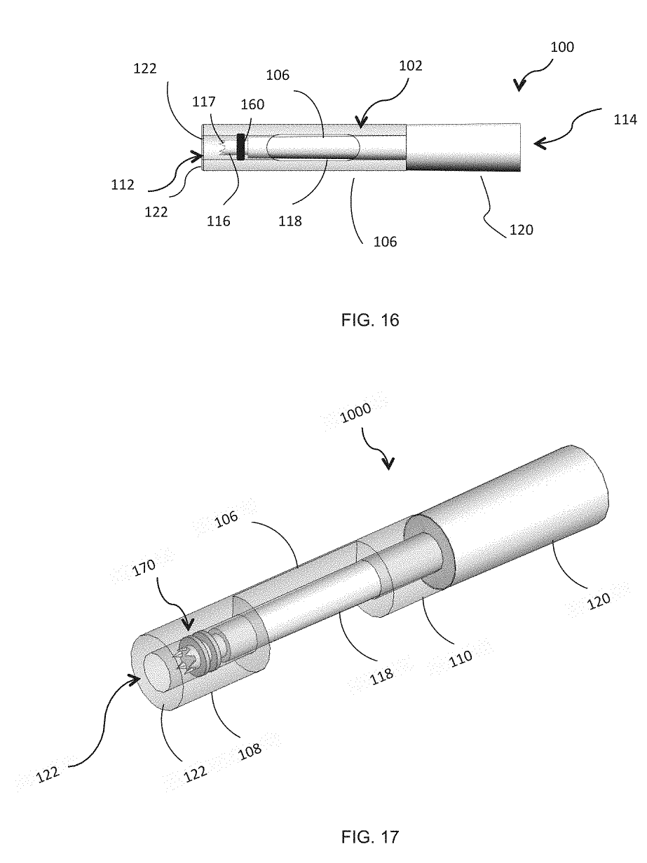

FIG. 16 is a side view of the allergy skin testing device 100 of FIG. 2, further including a sealing ring 160 seated in a circumferential notch in the sharp portion 116 for providing a sliding seal against the interior surface of the passageway 112 of the annular dull probe portion 108. The sealing ring 160 can be a substantially friction-free configuration of a sealing ring, such as an O-ring, or a piston seal with a z- or v-shaped cross-section. This type of sealing ring can be coated with a friction-free coating, such as Slik-Sil® from Surface Solutions Group, Chicago, Ill., or parylene types N, C, or D, internal lubricated silicone, or a dry lubricant, or similar coating to achieve substantially friction-free performance that facilitates easy and smooth longitudinal movement of the sharp portion 116 of the central post 114 within the passageway 112 of the annular dull probe portion 108. Alternatively, the sealing ring 160 can be used without a friction-free coating. The sealing ring 160 ensures that the sharp probes 117 will contact the skin within an area that is substantially radially equidistant from the annular surface 122 of the dull probe portion 108, resulting in a more consistent surrounding pain gate effect. Without a sealing ring 160, even slight lateral movements of the device 100 while pressing the device 100 against the skin can result in the sharp probes 117 contacting the skin off-center with respect to the annular surface 122 of the dull probe portion 108, possibly resulting in a diminished surrounding pain gate effect for at least some of the sharp probes 117.

FIG. 17 is a translucent isometric view of the alternate embodiment 1000 of the skin testing device having a resilient compressible portion 104 with only a single bendable element 106, also including a pair of sealing rings 170 each seated in a respective circumferential notch in the sharp portion 116. The pair of sealing rings 170 provide a stabilizing sliding seal against the interior surface of the passageway 112 of the annular dull probe portion 108. The pair of sealing rings 117 provide added stability against bending forces due to the asymmetric resilient compressible portion 104 having only a single bendable element 106 configured to resiliently bend when compressed as the device is pressed against the skin. By absorbing at least some of the bending forces due to compression, the sharp probes 117 are more likely to contact the skin within an area that is radially equidistant from the annular surface 122 of the dull probe portion 108, resulting in a more consistent surrounding pain gate effect. As in the embodiment of FIG. 16, the pair of sealing rings 170 can be coated with a friction-free coating.

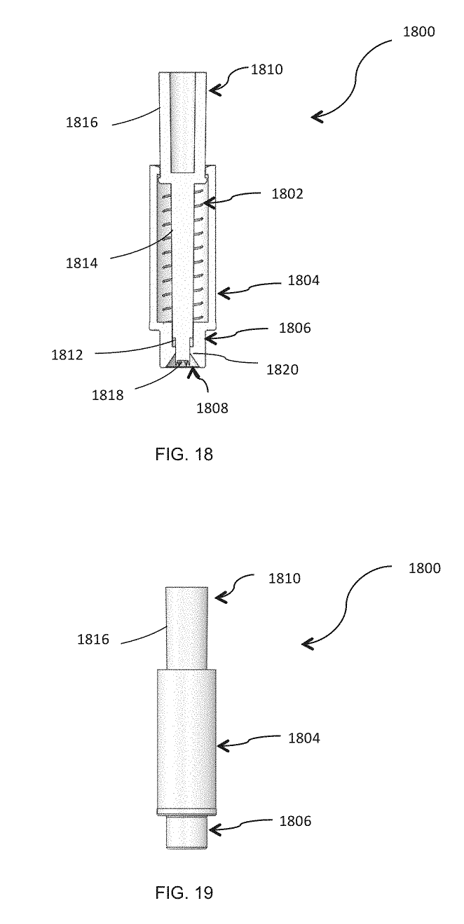

FIG. 18 is a cross-sectional side view of yet another alternate embodiment 1800 of the skin testing device having a spring 1802 serving as the resilient compressible portion, and a cover portion 1804 that extends over the spring 1802. The cover portion 1804 extends from the annular dull probe portion 1806 over the spring 1802.

Extending through a passageway 1808 is a central post 1810 having a sharp portion 1812, a longitudinal portion 1814, and a guide portion 1816. The sharp portion 1812 has one or more sharp probes 1818 extending longitudinally and being configured to administer an allergen to the skin.

Note that in this embodiment 1800, the sharp portion 1812 is maintained in concentric alignment with the annular dull probe portion 1806 by the narrowing 1820 having a slightly larger diameter than the diameter of the sharp portion 1812, such that they are in concentric sliding contact with each other. A friction-free coating, such as Slik-Sil® from Surface Solutions Group, Chicago, Ill., or parylene types N, C, or D, internal lubricated silicone, or a dry lubricant, or similar coating, can be used to achieve substantially friction-free relative movement.

FIG. 19 is a side view of the embodiment of FIG. 18, showing the annular dull probe portion 1806 contacting the skin before downward force is applied to the guide portion 1816. Downward force applied to the guide portion 1816 causes the entire central post 1810 to move downward and into the cover portion 1804, thereby compressing the spring 1802 inside, which in turn urges the annular dull probe portion 1806 to press into the skin surrounding the skin that is impinged upon by the sharps 1818, thereby activating a surrounding pain gate effect while the sharps introduce allergen to the skin.

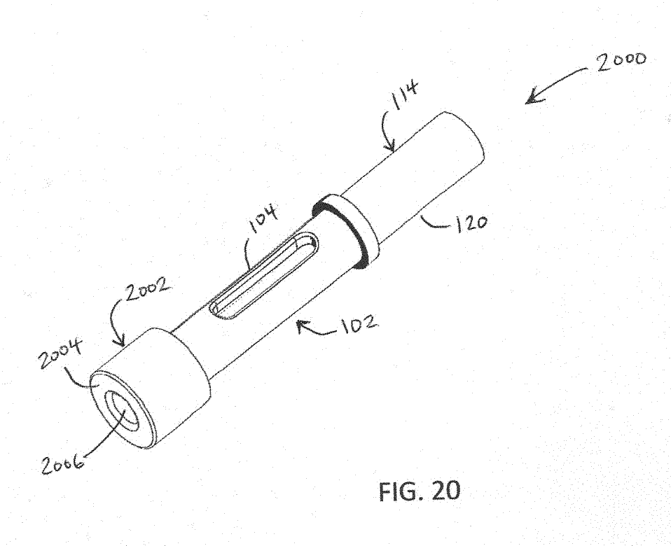

With reference to FIG. 20, an isometric view of an embodiment of a single allergy skin testing device 2000 is shown, the device 2000 having a rigid annular pain-reduction structure 2002 supported by a compressible structure 102 (also called a compressible annular pain-reduction structure 102 in the discussion of FIGS. 1 and 2, above) having a resilient (e.g., springy or spring-like) compressible portion 104. Concentrically extending into and attached to the compressible structure 102 is the central post 114 having a guide portion 120 for guiding the device 2000 towards the skin of a patient to be tested. The annular skin contacting surface 2004 of the rigid annular pain-reduction structure 2002 is a generally flat surface that is the first feature of the device 2000 that presses against the skin.

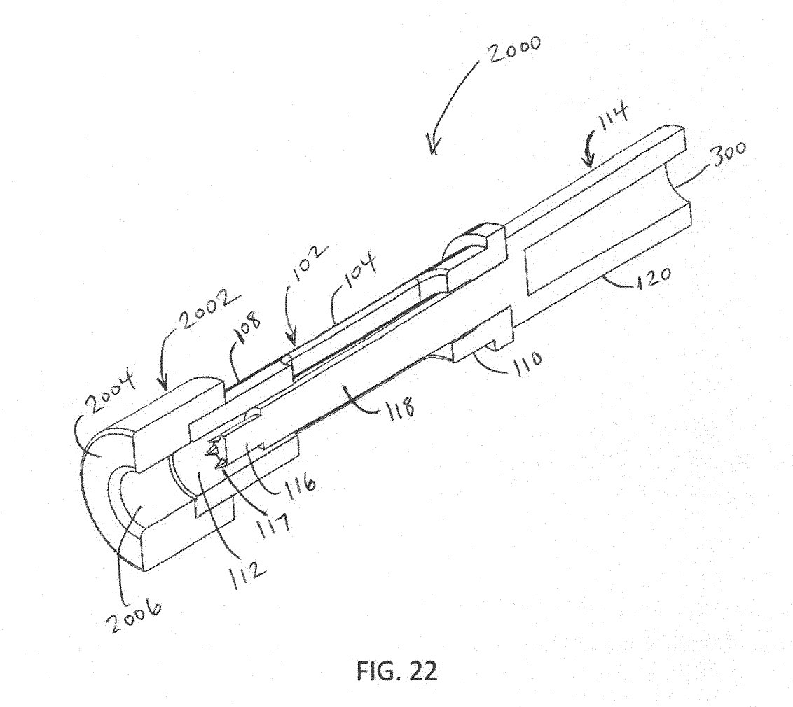

Due to the compressibility of the compressible structure 102, the rigid annular pain-reduction structure 2002 supported by the compressible structure 102 is movable with respect to the central post 114, and therefore the rigid annular pain-reduction structure 2002 is movable with respect to the one or more sharp probes 117 of the sharp portion 116 of the central post 114, as shown in FIG. 22.

Also, since the rigid annular structure 2002 is movable, there is a time delay between the contact made by the annular skin contacting surface 2004 of the rigid annular pain-reduction structure 2002, and contact made by the sharp probes 117 of the sharp portion 116. During the time delay, the sharp probes 117 of the sharp portion 116 move through the rigid annular pain-reduction structure 2002 towards the skin, as the compressible structure 102 compresses so as to press the annular skin contacting surface 2004 of the rigid annular pain-reduction structure 2002 against the skin.

In other embodiments, the annular skin contacting surface 2004 can include surface features that depart from being flat, such as texture, ridges, bumps, and/or protrusions.

The annular skin contacting surface 2004 could also include a surface of revolution. A surface of revolution is a surface in Euclidean space created by rotating a curve (the generatrix) around an axis of rotation. A circle that is rotated about an axis that does not intersect the interior of a circle generates a torus which does not intersect itself (a ring torus), which is the general shape of a bagel, for example. A closed semi-circle (a half-circle closed by a straight line) rotated about the central axis of the rigid annular pain-reduction structure 2002 would generate a half of a ring torus, which would look like a half of a bagel, such as would result from slicing a bagel in half before making a sandwich, each half having a flat annular surface, each half also having a convex surface.

Thus, an embodiment of the annular skin contacting surface 2004 can have the shape of the convex side of a half ring torus. Another embodiment can be a half ring torus extending from the annular skin contacting surface 2004, but the half ring torus having a larger inner diameter and a smaller outer diameter than the annular skin contacting surface 2004, so that it forms a rounded ridge that runs around a central annular portion of the annular skin contacting surface 2004.

The half ring torus can be added to a substantially flat version of the annular skin contacting surface 2004 as a separate half ring torus glued or otherwise bonded to the annular skin contacting surface 2004, or it can be co-molded to provide a convex half ring torus end without need for adhesive or attachment features.

The convex surface of a half ring torus provides a soft inviting appearance to the surface of the device 2000 that will contact the skin, while also providing increased dullness that can enhance the surrounding pain gate effect of the rigid annular probe portion 2002 when pressed against the skin.

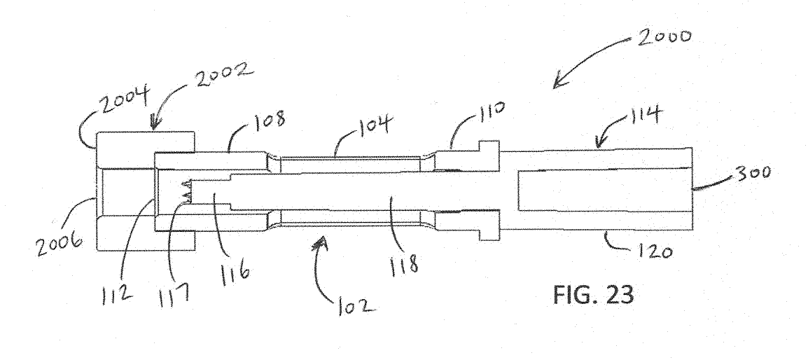

The rigid annular probe portion 2002 includes a second passageway 2006, through which the sharps 117 of the sharp portion 116 of the central post 114 (as shown in FIGS. 22 and 23) will emerge so as to press against the skin so as to deliver allergen to the skin for testing purposes.

The rigid annular probe portion 2002 can be made from similar or the same rigid material as used to make the central post 114. In other embodiments, the rigid annular probe portion 2002 is made from another material that is relatively “rigid” in that it is less compressible (i.e., having a greater Shore A Durometer) than the material of the compressible structure 102. In this preferred embodiment, the material used for the compressible structure 102 is silicone with a hardness between 30-40 Shore A Durometer.

In some embodiments, the rigid annular probe portion 2002 is larger in outer diameter, e.g., ⅜″ than the outer diameter, e.g., 2/8″ of the base portion 110 of the compressible structure 102. In some embodiments, the rigid annular probe portion 2002 has the same outer diameter, e.g., 2/8″ as the outer diameter, e.g., 2/8″ of the base portion 110 of the compressible structure 102.

The rigid annular probe portion 2002 can be made as a part that is distinct from the base portion 110 of the compressible structure 102, or can be co-molded as two materials that are joined together during the molding process without the need for adhesives or snapping together, for example.

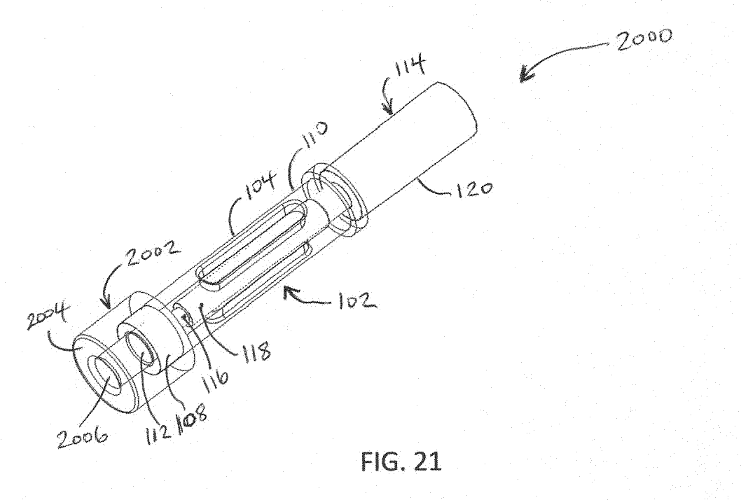

FIG. 21 is a wire-frame isometric view of the allergy skin testing device of FIG. 20, again showing the rigid annular pain-reduction structure 2002 supported by a compressible structure 102 having a resilient compressible portion 104, and a base portion 110 that is pushed by the guide portion 120 of the central post 114. The longitudinal portion 118 of the central post 114 concentrically extends through and is attached to the based portion 110 of the compressible structure 102.

The leading portion 108 of the compressible structure 102 has a first passageway 112 through which the sharp portion 116 and then the longitudinal portion 118 will pass as the sharp portion 116 moves towards the skin via the second passageway 2006 through the rigid annular pain-reduction structure 2002. Thus, the second feature of the device that presses against the skin, after the annular skin-contacting surface 2004, is the one or more sharp probes 117 of the sharp portion 116.

Referring to FIG. 22, a cut-away isometric view of the single allergy skin testing device 2000 of FIG. 20 is shown. The one or more sharp probes 117 of the sharp portion 116 are clearly visible in this view.

It is also clear in this view of the single allergy skin testing device 2000 that the central post 114 has a sharp portion 116, a longitudinal portion 118, and a guide portion 120. The sharp portion 116 has one or more sharp probes 117 extending longitudinally and being configured to administer an allergen to the skin, such as an allergen in solution into which at least the sharp probes 117 are dipped before being pressed against the skin. The guide portion 120 serves to facilitate guiding the sharp portion 116 towards the skin. The longitudinal portion 118 extends between the sharp portion 116 and the guide portion 120.

The compressible structure 102 has a leading portion 108 surrounding the sharp portion 116 of the central post 114, a resilient compressible portion 104, and a base portion 110. The resilient compressible portion 104 is configured to compress along the longitudinal portion of the central post 114 while the base portion 110 is urged towards the leading portion 108 by the guide portion 120. The leading portion 108 has a first passageway 112 that allows passage of the sharp portion 116 therethrough.

The rigid annular probe portion 2002 has an annular skin-contacting surface 2004, and a second passageway 2006 extending through the rigid annular probe portion 2002. The rigid annular probe portion 2002 extends longitudinally beyond the sharp portion 116 of the central post 114, before the resilient compressible portion 104 is compressed. The second passageway 2006 allows passage of the sharp portion 116 therethrough, while the resilient compressible portion 104 is compressed. The annular skin-contacting surface 2004 is configured as discussed with reference to FIG. 1 so as to activate a surrounding pain gate effect when pressed against the skin before and while the one or more sharp probes 117 are pressed against the skin so as to administer the allergen to the skin.

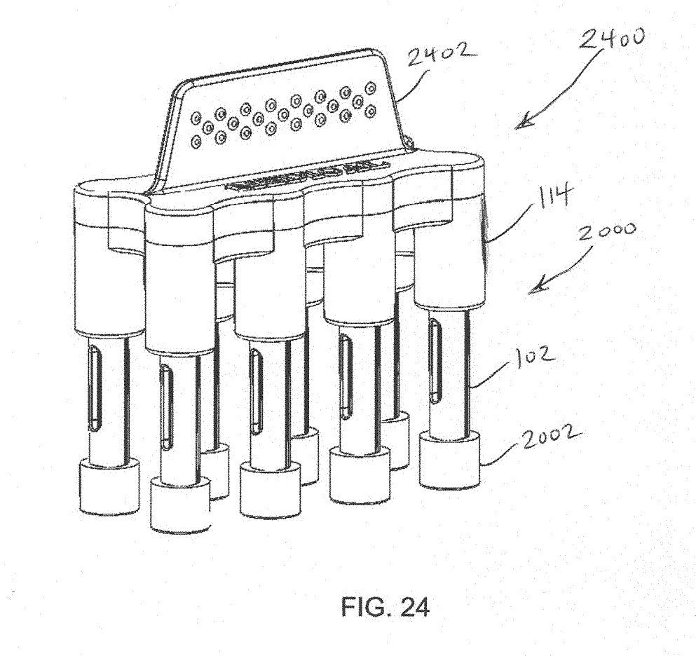

Also shown in FIG. 22 is an attachment feature 300 of the guide portion 120 of the central post 114. A single-test handle (not shown) for controlling the device 2000 can be attached to the device 2000 by inserting an attachment peg (not shown) of the single-test handle (not shown) into the attachment feature 300. Alternatively, one peg of a plurality of attachment pegs of a multiple test grip 2402 of a multiple test allergy skin testing device 2400, such as shown in FIG. 24 below, can be used to attach the device 2000 to a multiple test grip 2402 so as to assemble a multiple test device 2400, such as shown in FIG. 24.

FIG. 23 is a cut-away side view of the allergy skin testing device 2000 of FIG. 20, showing another view of all the elements discussed in FIG. 22.

FIG. 24 is an isometric view of a multiple test allergy skin testing device 2400 including a plurality of allergy skin testing devices 2000 of FIG. 20. As in FIG. 20, for example, each allergy skin testing device 2000 includes a central post 114, a compressible structure 102, and a rigid annular probe portion 2002. The plurality of allergy skin testing devices 2000 are joined together using a grip 2402, which has a plurality of attachment pegs (not shown).

In some embodiments, each compressible structure 102 included in a multiple test allergy skin testing device 2400 can be of a lesser stiffness then the compressible structure 102 for use in a single tester device 2000 used separately.

The multiple test allergy skin testing device 2400 is placed onto a multi-well allergen solution tray before performing an allergy test upon the skin of a patient. Each well of the multi-well allergen solution tray can contain a solution of a different allergen, such as pet dander, grass, tree pollen, and household dust, for example.

Each well of the multi-well allergen solution tray that is configured to be cooperative with the multiple test allergy skin testing device 2400 has an opening that is of slightly greater diameter than the diameter of the rigid annular probe portion 2002. The opening is the wide end of the surface of a right conic frustum (i.e., a cone truncated by two parallel planes perpendicular to the central axis of the cone), such that the inner walls of the truncated cone guide the rigid annular probe portion 2002 to the annular ledge at the bottom of the truncated cone, the diameter of the annular ledge being just slightly lesser diameter than the opening of the well, such that the rigid annular probe portion 2002 rests snugly at the bottom of the well.

Then, the grip 2402 can be used to push against the respective central post 114 of the respective testing device 2000 such that the sharp portion 116, having the one or more sharp probes 117 extending therefrom, moves through the first passageway 112, and then out through the second passageway 2006 so as to extend down into the well until a central stop extending upwards from the center of the well impacts upon the center of the end of the sharp portion 116. In the embodiment shown in FIG. 22, for example, the plurality of sharp probes 117 extend from the end of the sharp portion 116, and are arranged about the circumference of the end of the sharp portion 116. Consequently, there is a central area of the end of the sharp portion 116 that has no sharp probes 117, and can therefore be the area where the central stop impacts upon the end of the sharp portion 116 so as to stop further movement of the sharp portion 116 into the well.

Once thereby fully inserted into the well, the sharp probes 117 are immersed in the allergy solution of that particular well, such that when the device 2000 of the device 2400 is pulled out of the well, an effective quantity of allergen solution adheres to each of the sharp probes 117.

The central stop is of a length that prevents the sharp probes from impacting upon the bottom of the well, thereby reducing their sharpness. Thus, the central stop prevents damage to the plurality of sharp probes 117 when applying allergy solution using the wells configured to work cooperatively with the rigid annular probe portion 2002 of the device 2000.

Alternatively, the bottom of the well can be deep enough such that the sharps cannot reach the bottom, but nevertheless are immersed in allergen solution, thereby preventing damage to the plurality of sharp probes 117 when applying allergy solution.

In other embodiments, the device 2000 can include a rigid probe portion having a cross-section that is not annular, i.e., circular, instead of the rigid annular probe portion 2002, which has a circular ring cross-section. The rigid probe portion 2002 could instead have a cross section of another shape, such as one of the regular convex polygons: a triangle, a square, a pentagon, a hexagon, a heptagon, or an octagon, for example. Further, any regular convex polygon would work well, as would other shapes that could be inscribed in a circle, such as a rectangle. Thus, any of the regular polygonal shapes can provide an effectively “annular” shape for the cross-section of the rigid annular probe portion 2002. Alternatively, an ellipsoidal shape can also be used. Likewise, the compressible structure 102 can have a cross section of another shape, such as a triangle, a square, a pentagon, a hexagon, a heptagon, or an octagon, for example.

Further, although the compressible structure 102 is taught as having a resilient compressible portion 104 having a pair of bendable elements 106 as shown in FIG. 1, instead, there can be three, or four, or five, or six bendable elements, for example.

Other modifications and implementations will occur to those skilled in the art without departing from the spirit and the scope of the invention as claimed. Accordingly, the above description is not intended to limit the invention, except as indicated in the following claims.

What is claimed is:

1. An allergy skin testing device, the device to be used by pressing the device against skin, the allergy skin testing device comprising:
a central post having a sharp portion, a longitudinal portion, and a guide portion,
the sharp portion having one or more sharp probes extending longitudinally and being configured to administer an allergen to the skin,
the guide portion being configured to facilitate guiding the sharp portion towards the skin,
the longitudinal portion extending between the sharp portion and the guide portion;
a compressible structure having a leading portion surrounding the sharp portion of the central post, a resilient compressible portion, and a base portion,
the resilient compressible portion being configured to compress along the longitudinal portion of the central post while the base portion is urged towards the leading portion by the guide portion,
the leading portion having a first passageway to allow passage of the sharp portion therethrough; and
a rigid annular probe portion having an annular skin-contacting surface, and a second passageway extending through the rigid annular probe portion,
the rigid annular probe portion extending longitudinally beyond the sharp portion of the central post, before the resilient compressible portion is compressed,
the second passageway allowing passage of the sharp portion therethrough, while the resilient compressible portion is compressed,
the annular skin-contacting surface being configured to activate a surrounding pain gate effect when pressed against the skin before and while the one or more sharp probes are pressed against the skin so as to administer the allergen to the skin.

2. The device of claim 1, wherein the resilient compressible portion of the compressible structure includes: a plurality of bendable elements configured to resiliently bend when compressed.

3. The device of claim 1, wherein the resilient compressible portion of the compressible annular pain-reduction structure includes: a plurality of bendable elements configured to resiliently bend outwards when compressed.

4. The device of claim 1, wherein an inner diameter of the first passageway is substantially similar to an inner diameter of the second passageway.

5. The device of claim 1, wherein an outer diameter of the annular skin-contacting surface is greater than an outer diameter of the leading portion of the compressible structure.

6. The device of claim 1, wherein an outer diameter of the annular skin-contacting surface is substantially similar to an outer diameter of the leading portion of the compressible structure.

7. The device of claim 1, wherein the annular skin-contacting surface includes: one or more bumps configured to contact the skin so as to activate a surrounding pain gate effect when pressed against the skin.

8. The device of claim 1, wherein the annular skin-contacting surface of the rigid annular probe portion includes:
surface features that depart from being flat.

9. The device of claim 1, wherein the annular skin-contacting surface of the rigid annular probe portion includes a convex surface of revolution defined by rotating a generatrix curve about a central axis of the rigid annular pain-reduction structure, the generatrix curve being one of:
a semi-circle, a semi-ellipse, a convex half sine curve, a convex spline curve.

10. The device of claim 1, wherein the annular skin-contacting surface of the rigid annular probe portion includes:
a half ring torus surface as the annular skin-contacting surface,
thereby providing a rounded end of the of the rigid annular probe portion having enhanced dullness to augment the surrounding pain gate effect of the rigid annular probe portion.

11. A multiple test allergy skin testing device, the device to be used by pressing the device against skin, the multiple test allergy skin testing device comprising:
a plurality of central posts, each central post having a sharp portion, a longitudinal portion, and a guide portion,
the sharp portion having one or more sharp probes extending longitudinally and being configured to administer an allergen to the skin,
the guide portion being configured to facilitate guiding the sharp portion towards the skin,
the longitudinal portion extending between the sharp portion and the guide portion;
a plurality of compressible structures, each compressible structure having a leading portion surrounding the sharp portion of the central post, a resilient compressible portion, and a base portion,
the resilient compressible portion being configured to compress along the longitudinal portion of the central post while the base portion is urged towards the leading portion by the guide portion,
the leading portion having a first passageway to allow passage of the sharp portion therethrough;
a plurality of rigid annular probe portions, each rigid annular probe portion having an annular skin-contacting surface, and a second passageway extending through the rigid annular probe portion,
the rigid annular probe portion extending longitudinally beyond the sharp portion of the central post, before the resilient compressible portion is compressed,
the second passageway allowing passage of the sharp portion therethrough, while the resilient compressible portion is compressed,
the annular skin-contacting surface being configured to activate a surrounding pain gate effect when pressed against the skin before and while the one or more sharp probes are pressed against the skin so as to administer the allergen to the skin; and
a gripping element, connected to each guide portion of each of the plurality of central posts, the gripping element being for pressing each rigid annular probe portion against the skin, and then pressing the sharp portion of each respective central post against the skin, using one press of the gripping element.

12. The device of claim 11, wherein the resilient compressible portion of each compressible structure includes: a plurality of bendable elements configured to resiliently bend when compressed.

13. The device of claim 11, wherein the resilient compressible portion of each compressible structure includes: a plurality of bendable elements configured to resiliently bend outwards when compressed.

14. The device of claim 11, wherein an inner diameter of each first passageway is substantially similar to an inner diameter of each respective second passageway.

15. The device of claim 11, wherein an outer diameter of each annular skin-contacting surface is greater than an outer diameter of the leading portion of each respective compressible structure.

16. The device of claim 11, wherein an outer diameter of each annular skin-contacting surface is substantially similar to an outer diameter of each respective leading portion of the compressible structure.

17. The device of claim 11, wherein each annular skin-contacting surface includes one or more bumps configured to contact the skin so as to activate a surrounding pain gate effect when pressed against the skin.

18. The device of claim 11, wherein each annular skin-contacting surface of each rigid annular probe portion includes: surface features that depart from being flat.

19. The device of claim 11, wherein each annular skin-contacting surface of each rigid annular probe portion includes a convex surface of revolution defined by rotating a generatrix curve about a central axis of the rigid annular pain-reduction structure, the generatrix curve being one of:
a semi-circle, a semi-ellipse, a convex half sine curve, a convex spline curve.

20. The device of claim 11, wherein each annular skin-contacting surface of each rigid annular probe portion includes:
a half ring torus surface as the annular skin-contacting surface, thereby providing a rounded end of the of the rigid annular probe portion having enhanced dullness to augment the surrounding pain gate effect of the rigid annular probe portion.
